(19) United States
(12) Patent Application Publication
(10) Pub. No.: US 2016/0345943 A1
(43) Pub. Date: Dec. 1, 2016

(54) MEDICAL CLOSURE SYSTEM

(71) Applicant: LEPU MEDICAL TECHNOLOGY (BEIJING) CO., LTD, Beijing (CN)

(72) Inventors: Kai Cui, Beijing (CN); Chang Li, Beijing (CN); Hui Li, Beijing (CN); Ming Yang, Beijing (CN); Xia Zhang, Beijing (CN)

(73) Assignee: LEPU MEDICAL TECHNOLOGY (BEIJING) CO., LTD, Beijing (CN)

(21) Appl. No.: 15/117,752

(22) PCT Filed: Apr. 30, 2014
(86) PCT No.: PCT/CN2014/076686

§ 371 Date: Aug. 10, 2016

(51) Int. Cl. A61B 17/00

(52) U.S. Cl. CPC A61B 17/0057; A61B 2017/00606; A61B 2017/00004; A61B 2017/00597; A61B 2017/00623; A61B 2017/00991

(57) ABSTRACT

A medical closure system comprises a closure stent (100) and a delivery device (200); the closure stent (100) comprises a stent outer layer (1), a film (3) disposed on the stent outer layer (1), and a mandrel (8) passing through the stent outer layer (1); the distal end of the mandrel (8) is fixedly connected to the distal end of the stent outer layer (1); the closure stent (100) is made of a degradable polymer; the delivery device (200) comprises an external push tube (11) and an internal push rod (10) installed in the external push tube (11); the external push tube (11) and the internal push rod (10) are movable relative to each other in an axial direction; the external push tube (11) is movably connected to the proximal end of the stent outer layer (1); and the internal push rod (10) is movably connected to the proximal end of the mandrel (8). The medical closure system can automatically degrade after being implanted into a human body, thus eliminating the hidden risk of a side effect caused by residue of the closure stem left in the human body.

### TECHNICAL FIELD

The present invention relates to the field of medical instruments, and particularly relates to a medical closure system.

### BACKGROUND

The interventional therapy of congenital heart disease has become quite common, and a closure system is a main instrument for the interventional therapy. The closure system mainly includes a closure stent and a delivery device. At present, many companies at home and abroad launch products to the market; however the closure stents of these products are all formed by braiding nickel-holmium memory alloy wires and then performing heat-setting, and the self-expandable closure stents are manufactured by using the super-elasticity of nickel-holmium memory. However, the human body does not need the instrument for a long term, the internal membrane is completely covered within about three months, and then the closure stent becomes a useless foreign matter and is accompanied by various side effects, e.g., nickel ions are released to damage human tissues, etc. In addition, degradable material closure stents manufactured by adopting an existing platform easily retracts, so that the clamping force is insufficient and the operation fails.

### SUMMARY

### (I) Technical Problems to be Solved

The object of the present invention is to provide a medical closure system for solving the problems that a closure stent automatically retracts and is difficult to degrade.

### (II) Technical Solutions

The present invention provides a medical closure system, including a closure stent and a delivery device, wherein the closure stent includes a stent outer layer, a film disposed on the stent outer layer, and a mandrel passing through the stent outer layer; the distal end of the mandrel is fixedly connected to the distal end of the stent outer layer; the closure stent is made of a degradable polymer; the delivery device includes an external push tube and an internal push rod installed in the external push tube; the external push tube and the internal push rod are movable relative to each other in an axial direction; the external push tube is movably connected to the proximal end of the stent outer layer; and the internal push rod is movably connected to the proximal end of the mandrel.

A clamping ring is disposed at the proximal end of the mandrel.

Preferably, the clamping ring is a boss formed at the outer edge of the proximal end of the mandrel.

A stent waist is disposed on the stent outer layer; when the stent outer layer is spread, an upper disc surface is formed between the distal end of the stent outer layer and the stent waist; and a lower disc surface is formed between the proximal end of the stent outer layer and the stent waist.

The stent waist is connected to the proximal end of the stent outer layer and the distal end of the stent outer layer respectively by multiple stent beams; the film is disposed between any two adjacent stent beams; and a bent portion is formed in the middle of each stent beam.

The stent waist has a telescopic structure, and the length and the diameter thereof can be determined as required.

The proximal end and the distal end of the stent waist are connected to each other by multiple waist beams.

The stent outer layer is a braided tube braided with helical cross ribbons, and the stent waist is disposed at the middle section of the braided tube.

The internal push rod is provided with a connection guiding portion for connection with the mandrel, and a guiding hole matched with the connection guiding portion is formed in the proximal end of the mandrel.

The delivery device further includes an external sheath, and the external push tube is installed in the external sheath and is movable axially along the external sheath.

### (III) Beneficial Effects

According to the medical enclosure system provided by the present invention, the closure stent is delivered to a therapeutic position by the delivery device, and the stent outer layer is spread to separate from the delivery device, so that the closure stent is released to the therapeutic position to accomplish closure operation and is prevented from returning to the original position; the closure stent of the present invention is made of a degradable polymer, and can automatically degrade after the medical enclosure system is implanted into a human body, thus eliminating the hidden risk of a side effect caused by residue of the closure stem left in the human body.

### BRIEF DESCRIPTION OF THE DRAWINGS

FIG. 1 is a three-dimensional structural diagram in embodiment 1 of the present invention;

FIG. 2 is a view along the axial end of a closure stent in embodiment 1 of the present invention;

FIG. 3 is a front view of the closure stent in a non-spread state in embodiment 1 of the present invention;

FIG. 4 is a stereogram without a film in embodiment 1 of the present invention;

FIG. 5 is a front view in embodiment 2 of the present invention;

FIG. 6 is a front view of the closure stent in a non-spread state in embodiment 2 of the present invention;

FIG. 7 is a stereogram in embodiment 3 of the present invention.

Reference signs: 1: stent outer layer; 2: upper disc surface; 3: film; 4: lower disc surface; 5: stent waist; 6: stent beam; 7: clamping ring; 8: mandrel; 9: connection guiding portion; 10: internal push rod; 11: external push tube; 12: external sheath; 13: bent portion; 100: closure stent; 200: delivery device.

### DETAILED DESCRIPTION OF THE PREFERRED EMBODIMENTS

The specific embodiments of the present invention will be further described below in combination with the accompanying drawings and embodiments.

### Embodiment 1

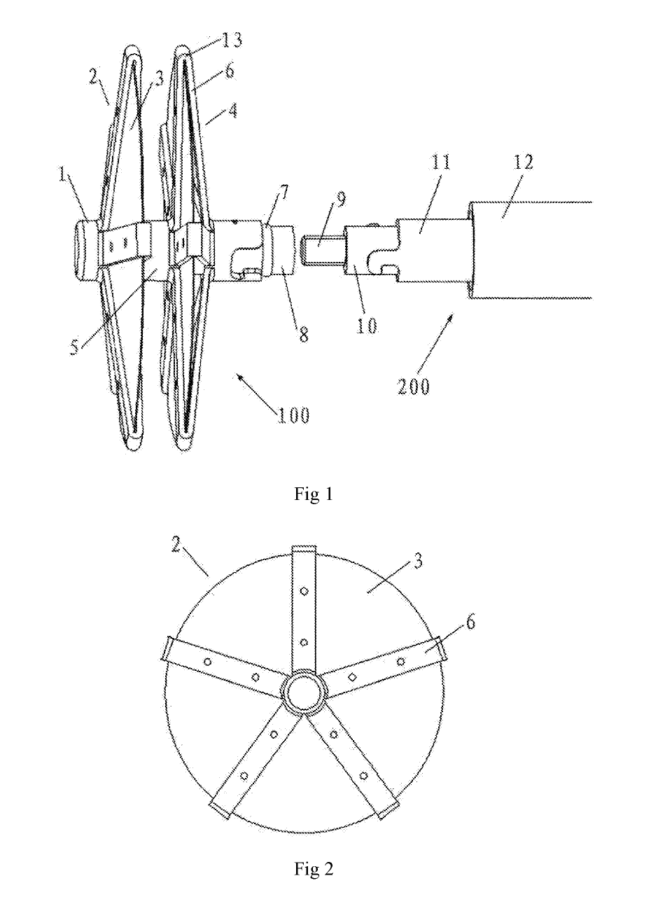

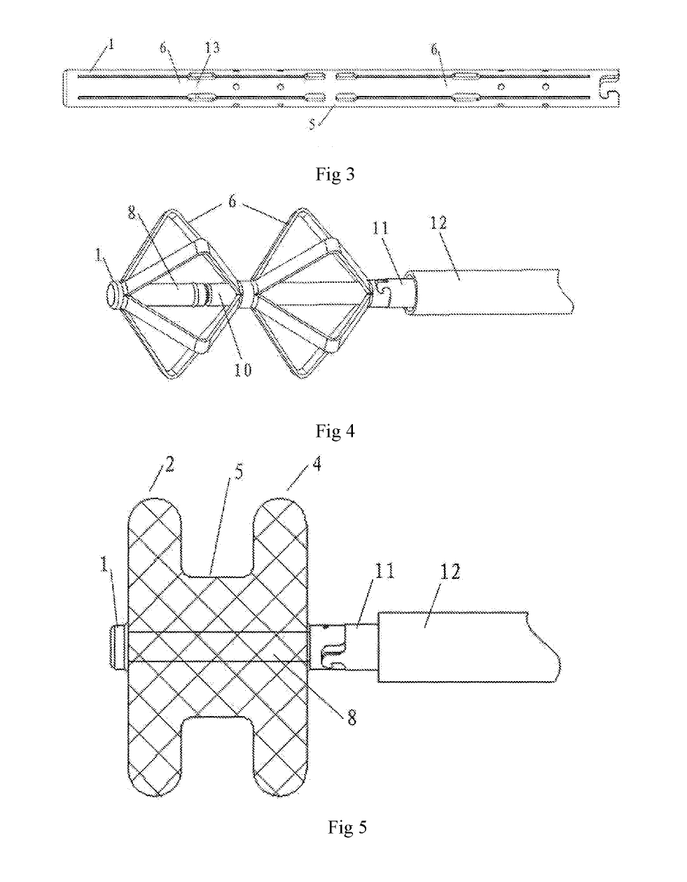

With reference to FIGS. 1 to 4, a medical closure system in the present invention includes a closure stent 100 and a delivery device 200, wherein the closure stent 100 includes a stent outer layer 1, a film 3 disposed on the stent outer layer 1, and a mandrel 8 passing through the stent outer layer 1. The stent outer layer 1 can be spread, so that the film 3 disposed on the stent outer layer 1 is spread to form a disc surface structure, and the spread closure stent 100 has a closing function. The distal end of the mandrel 8 is fixedly connected to the distal end of the stent outer layer 1. On the closure stent 100, one end, fixed to the stent outer layer 1, of the mandrel 8 is referred to as the distal end (with reference to the left end in FIG. 1), while the other end is referred to as the proximal end (with reference to the right end in FIG. 1). The closure stent 100 is made of a degradable polymer. The degradable polymer may be silk fibroin, PLA (polylactic acid), PGA (polyglycolic acid), PGLA (poly glycolide lactide) or composite material, etc. The delivery device 200 includes an external sheath 12, an external push tube 11 and an internal push rod 10 installed in the external push tube 11; the external push tube 11 and the internal push rod 10 can are movable relative to each other in an axial direction; the external push tube 11 is movably connected to the proximal end of the stent outer layer 1; the internal push rod 10 is movably connected to the proximal end of the mandrel 8; and the external push tube 11 is installed in the external sheath 12, and the external push tube 11 is movable axially along the external sheath 12. The proximal end of the stent outer layer 1 can be connected with the external push tube 11 in any controllable connection mode such as a rotation mode.

In use, the internal push rod 10 is connected to the proximal end of the mandrel 8, the external push tube 11 is connected to the proximal end of the stent outer layer 1 and moves axially along the internal push rod 10, then the proximal end of the stent outer layer 1 moves to the distal end, and the stent outer layer 1 is deformed and spread according to a preset trend path to form a disc surface structure, thus achieving the purpose of deformation of the whole closure stent finally. Through operating the external push tube 11 and the internal push rod 10, the external push tube 11 and the internal push rod 10 separate from the stent outer layer 1 and the mandrel 8 respectively, so that the closure stent 100 is released to a specified therapeutic position; and the closure stent can also be recovered according to needs.

According to the medical enclosure system, the closure stent is delivered to a therapeutic position by the delivery device, and the stent outer layer is spread to separate from the delivery device, so that the closure stent is released to the therapeutic position to accomplish closure operation, so the use is convenient, and the closure stent is prevented from returning to the original position. The closure stent of the present invention is made of a degradable polymer, and can automatically degrade after the closure stent 100 is implanted into a human body, thus eliminating the hidden risk of a side effect caused by residue of the closure stem left in the human body in the prior art.

Further, a clamping ring 7 is disposed at the proximal end of the mandrel 8. The mandrel 8 may be a column of which the cross section is regularly polygonal, elliptical or circular. In this embodiment, preferably, the mandrel 8 is a cylinder, and the clamping ring 7 is a boss formed at the outer edge of the proximal end of the mandrel 8. The purpose of the clamping ring 7 is to ensure that the proximal end of the stent outer layer 1 can be powerfully constrained when arriving at the corresponding position of the mandrel, and to ensure that the spread stent outer layer cannot rebound freely but keeps a closure state.

Further, a stent waist 5 is disposed on the stent outer layer 1; when the stent outer layer 1 is spread, an upper disc surface 2 is formed between the distal end of the stent outer layer 1 and the stent waist 5; and a lower disc surface 4 is formed between the proximal end of the stent outer layer 1 and the stent waist 5. The stent waist 5 is preferably disposed at the middle section of the stent outer layer 1.

Further, the stent waist 5 is connected to the proximal end of the stent outer layer 1 and the distal end of the stent outer layer 1 respectively by multiple stent beams 6; the film 3 is disposed between any two adjacent stent beams 6; and a bent portion 13 is formed in the middle of each stent beam 6. When the external push tube 11 drives the proximal end of the stent out layer to move, the stent beams 6 are bent along the bent portions 13, and the film 3 is spread to form a disc surface structure. The stent outer layer 1 can be formed by engraving a whole tube or by welding strips or wires.

Further, in order to ensure that the closure stent can be expanded to the length and the diameter required for therapy, the stent waist 5 may have a telescopic structure, and the length and the diameter thereof can be determined as required. The stent waist 5 may have a netlike structure.

Further, any controllable connection mode such as threaded connection or clamping may be adopted between the mandrel 8 and the internal push rod 10. When the threaded connection is selected, the internal push rod 10 can rotate along a radial direction of the external push tube 11. In this embodiment, preferably, the internal push rod 10 is provided with a connection guiding portion 9 for connection with the mandrel 8, and a guiding hole (not shown in the figures) matched with the connection guiding portion 9 is formed in the proximal end of the mandrel 8. By connecting the connection guiding portion 9 with a buckle seat, the connection accuracy between the internal push rod 10 and the mandrel 8 is improved, and the internal push rod 10 is prevented from deviating from the mandrel 8 to cause operation failure.

Further, the delivery device further includes a handle (not shown in the figures), which is used for controlling the axial movement of the internal push rod 10 and the external push tube 11 for therapy.

### Embodiment 2

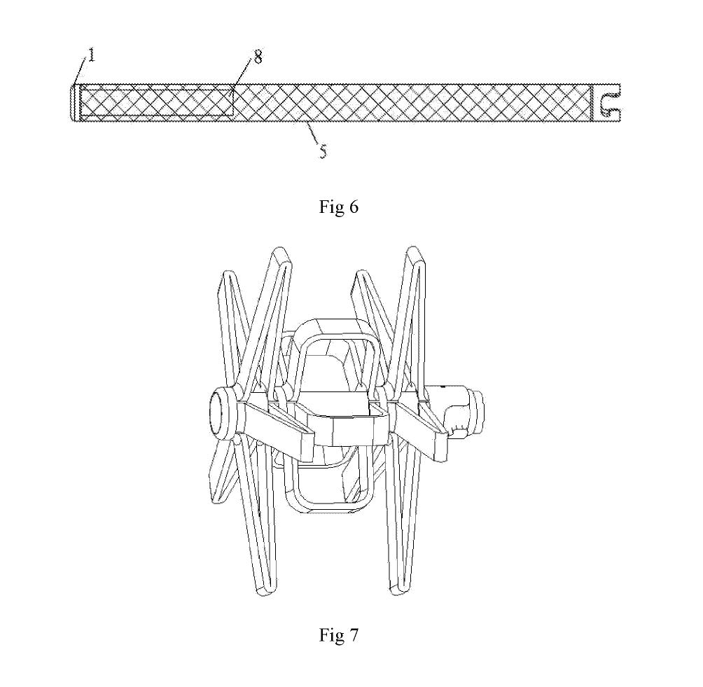

This embodiment is substantially the same as embodiment 1, and the difference lies in that: with reference to FIGS. 5 and 6, the stent outer layer 1 of the closure stent 100 is a braided tube braided with helical cross ribbons, and the stent waist 5 is disposed at the middle section of the braided tube with certain deformation function.

### Embodiment 3

This embodiment is substantially the same as embodiment 1, and the difference lies in that: as shown in FIG. 7, the proximal end and the distal end of the stent waist are connected to each other by multiple waist beams, and when the external push tube pushes the proximal end of the stent outer layer to move along the internal push rod, the waist beams are spread outwards to shorten the stent waist.

The retraction of the closure stent in the present invention means that before the mandrel is separated from the internal push rod, the external push tube and the proximal end of the stent outer layer may be separated from or reconnected to each other, and the deformed closure stent is retracted to have a strip or tube shape again and then retracted into the external sheath; the release of the closure stent means that after the proximal end of the stent outer layer arrives at the designed position, the proximal end of the stent outer layer is separated from the external push tube first, and then the proximal end of the mandrel is separated from the internal push rod, so that the closure stent is retained at the destination position.

### INDUSTRIAL APPLICABILITY

The present invention provides a medical closure system, including a closure stent and a delivery device, wherein the closure stent includes a stent outer layer, a film disposed on the stent outer layer, and a mandrel passing through the stent outer layer; the distal end of the mandrel is fixedly connected to the distal end of the stent outer layer; the closure stent is made of a degradable polymer; the delivery device includes an external push tube and an internal push rod installed in the external push tube; the external push tube and the internal push rod are movable relative to each other in an axial direction; the external push tube is movably connected to the proximal end of the stent outer layer; and the internal push rod is movably connected to the proximal end of the mandrel. According to the present invention, the closure stent can be delivered to a therapeutic position by the delivery device, and the stent outer layer is spread to separate from the delivery device, so that the closure stent is released to the therapeutic position to accomplish closure operation and is prevented from returning to the original position; the closure stent is made of a degradable polymer, and can automatically degrade after the closure stent is implanted into a human body, thus eliminating the hidden risk of a side effect caused by residue of the closure stem left in the human body.

What is claimed is:

1. A medical closure system, comprising a closure stent and a delivery device, wherein the closure stent comprises a stent outer layer, a film disposed on the stent outer layer, and a mandrel passing through the stent outer layer; the distal end of the mandrel is fixedly connected to the distal end of the stent outer layer; the closure stent is made of a degradable polymer; the delivery device comprises an external push tube and an internal push rod installed in the external push tube; the external push tube and the internal push rod are movable relative to each other in an axial direction; the external push tube is movably connected to the proximal end of the stent outer layer; and the internal push rod is movably connected to the proximal end of the mandrel.

2. The medical closure system of claim 1, wherein a clamping ring is disposed at the proximal end of the mandrel.

3. The medical closure system of claim 2, wherein the clamping ring is a boss formed at the outer edge of the proximal end of the mandrel.

4. The medical closure system of claim 1, wherein a stent waist is disposed on the stent outer layer; when the stent outer layer is spread, an upper disc surface is formed between the distal end of the stent outer layer and the stent waist; and a lower disc surface is formed between the proximal end of the stent outer layer and the stent waist.

5. The medical closure system of claim 4, wherein the stent waist is connected to the proximal end of the stent outer layer and the distal end of the stent outer layer respectively by multiple stent beams; the film is disposed between any two adjacent stent beams; and a bent portion is formed in the middle of each stent beam.

6. The medical closure system of claim 4, wherein the stent waist has a telescopic structure, and the length and the diameter thereof can be determined as required.

7. The medical closure system of claim 6, wherein the proximal end and the distal end of the stent waist are connected to each other by multiple waist beams.

8. The medical closure system of claim 1, wherein the stent outer layer is a braided tube braided with helical cross ribbons, and the stent waist is disposed at the middle section of the braided tube.

9. The medical closure system of claim 1, wherein the internal push rod is provided with a connection guiding portion for connection with the mandrel, and a guiding hole matched with the connection guiding portion is formed in the proximal end of the mandrel.

10. The medical closure system of claim 1, wherein the delivery device further comprises an external sheath, and the external push tube is installed in the external sheath and is movable axially along the external sheath.
